(19) United States
(12) Patent Application Publication
(10) Pub. No.: US 2025/0225959 A1
(43) Pub. Date: Jul. 10, 2025

(54) A METHOD OF FORMING THE NECK OF A STRINGED INSTRUMENT

(71) Applicant: R-Go Instruments Oy, TAMPERE (FI)

(72) Inventors: Pekka MÄKINEN, TAMPERE (FI); Jarkko RUOHOTIE, TAMPERE (FI)

(73) Assignee: R-Go Instruments Oy, TAMPERE (FI)

(21) Appl. No.: 18/850,785

(22) PCT Filed: Mar. 31, 2023
(86) PCT No.: PCT/FI2023/050180

§ 371 Date: Sep. 25, 2024

(30) Foreign Application Priority Data
Mar. 31, 2022 (FI) 20225278

(51) Int. Cl. G10D 1/08; G06T 7/60

(52) U.S. Cl. CPC G10D 1/08; G06T 7/60; G06T 2207/30196

(57) ABSTRACT

A method for shaping a neck of a stringed instrument, wherein measuring the musician's palm length in the direction of the forearm from the wrist's folding point to the plane of the thumb's base; measuring the musician's thumb length from the ulnar base crease to the thumb's tip plane; measuring the musician's thumb distance from the forefinger substantially in the direction of the musician's arm; measuring the thumb's relaxed point after the combined flexion-abduction movement; measuring the thumb's relaxed point after the combined extension-abduction movement; measuring in the direction of the forearm the distance from the thumb's base to the plane of the creases of the chord-playing fingers' base joints; measuring in the direction of the forearm the distance from the plane of the creases of the chord-playing fingers' base joints to the creases of the fingers where the fingers visibly separate from each other outside the rest of the palm; calculating the range of variation of the plane of the fretboard of the neck of the stringed instrument on the plane of the palm; calculating the range of variation of the angle of the fretboard of the neck of the stringed instrument on the plane of the palm in the depth direction.

### BACKGROUND OF THE INVENTION

The invention relates to a neck of a stringed instrument and particularly to a method for forming the neck of a stringed instrument. A musician finds a neck of suitable dimensions and a suitable profile by trying various stringed instruments having different sizes and different cross-sectional profiles. This typically requires using the instrument for a test period.

A problem of the above arrangement is that there is a large number of possible dimensions and profile shapes and their testing requires the fitting and test use of physical model pieces or stringed instruments.

### BRIEF DESCRIPTION OF THE INVENTION

It is thus the object of the invention to provide a novel method for forming the neck of a stringed instrument. The solution according to the invention is characterised by what is disclosed in the independent claims. Some preferred embodiments of the invention are disclosed in the dependent claims.

A stringed instrument, which includes a neck, is played such that the musician's the so-called chord-playing hand settles on the back surface of the instrument's neck such that the chord-playing hand's fingers extend to the front surface of the instrument's neck where the musician can press different points of the strings having been laid on the front side of the neck. It is important for the musician that the dimensioning and forming of the neck is done such that the musician's chord-playing hand settles in a position which does not strain the chord-playing hand and, on the other hand, the musician's individual dimensions have been taken into account.

The different movements of the hand's joints have their own limit values the exceeding of which requires active muscular work to maintain the joint's state. Within the limit values, the joint's state passively stays as is which enables a more relaxed playing feel. Relaxed points revealed through the relaxation of joint positions requiring active muscle work are important when shaping the neck profile. The invention is based on that fact that the definition of the neck profile of a stringed instrument is formed of the limit values and areas of the relaxed operation of the hand joints' positions and hand movements. By means of a profile measurer designed to support the measuring method, accurate measurement results tied to each other are obtained always at the same stage. Alternatively, it is possible to support the measuring method by a camera-based method where required dimensions are obtained by shooting images of the chord-playing hand's different positions by using a reference measure in the images, by means of which, the measurement results can be normalised. These results are utilised to provide an individual neck profile which caters for the musician's anatomy and physiology.

### BRIEF DESCRIPTION OF THE DRAWINGS

The invention will now be described in closer detail in connection with some embodiments and with reference to the accompanying drawings, in which:

FIG. 1a shows a typical stringed instrument;

FIGS. 1b-1d show various cross-sectional profiles (C type, V type, U/D type, symmetrical, asymmetrical) of a neck of a stringed instrument;

FIG. 2 shows a typical chord-playing position of a stringed instrument of the guitar type on the neck of the instrument;

FIG. 3 shows a side view of the stringed instrument of the guitar type and the “forward-backward” angle of the instrument in relation to a musician;

FIG. 4-a shows a chord-playing hand in the zero position;

FIGS. 4b and 4c show a plane of creases of chord-playing hand's base joints and a plane of creases of fingers' base members from different angles;

FIGS. 5a, 5b, 5c and 5d show a position of the thumb of the chord-playing hand in four different relaxed points and a rectangle formed by these points;

FIG. 5e shows dimensions of the chord-playing hand;

FIG. 6 shows a profile measurer and the chord-playing hand;

FIG. 7 shows parts of the profile measurer detached;

FIG. 8 shows a top view of the profile measurer;

FIG. 9 shows a side view of the profile measurer and the chord-playing hand;

FIG. 10 shows a diagonal top view of the profile measurer;

FIG. 11 shows a method where the chord-playing hand is shot by the camera with a measuring scale and/or a reference measure;

FIG. 12 is a schematic view of a cross-section of the neck and the significance of the different dimensions in relation to the neck;

FIG. 13 shows different positions of a fretboard in relation to the palm and particularly the folding point OT2 of the chord-playing fingers;

FIG. 14 shows positions of the wrist in the zero position, in flexion and in extension.

### DETAILED DESCRIPTION OF THE INVENTION

A stringed instrument is an instrument with its vibrant material being a wire-like tensed string. Most stringed instruments include several strings which are tuned up to different pitches. The sound of the instrument is created by the vibration of the string. A sound box intensifies the sound of the string. Built-in stringed instruments are constructed by a string body and a sound board fixedly integrated to each other.

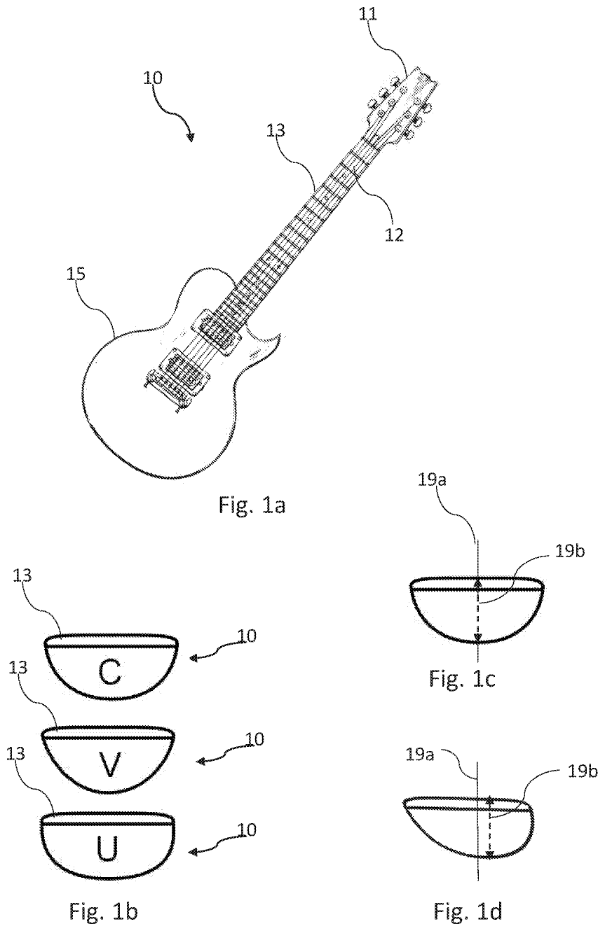

Parts of a stringed instrument 10 are shown in FIG. 1a. The stringed instrument 10 includes a fretboard 13, against which, the vibrant length of a string 12 can be shortened. Typically, the strings 12 are pressed by the fingers of a chord-playing hand 14. Such stringed instruments are e.g. an acoustic guitar, an electric guitar, a bass guitar, a violin, a viola, a cello, a double bass, a sitar, a balalaika, a lute, and a banjo. The profile of a neck 11 of the stringed instrument 10 can have various types of shapes. FIG. 1b shows three examples of different profile shapes which types are called, inter alia, C-, V- and U-type profiles. The U-type profile is also called a D)-type profile. The profile can be symmetrical (FIG. 1c) in relation to a longitudinal centre line 19a of the neck or asymmetrical (FIG. 1d) in relation to said centre line 19a.

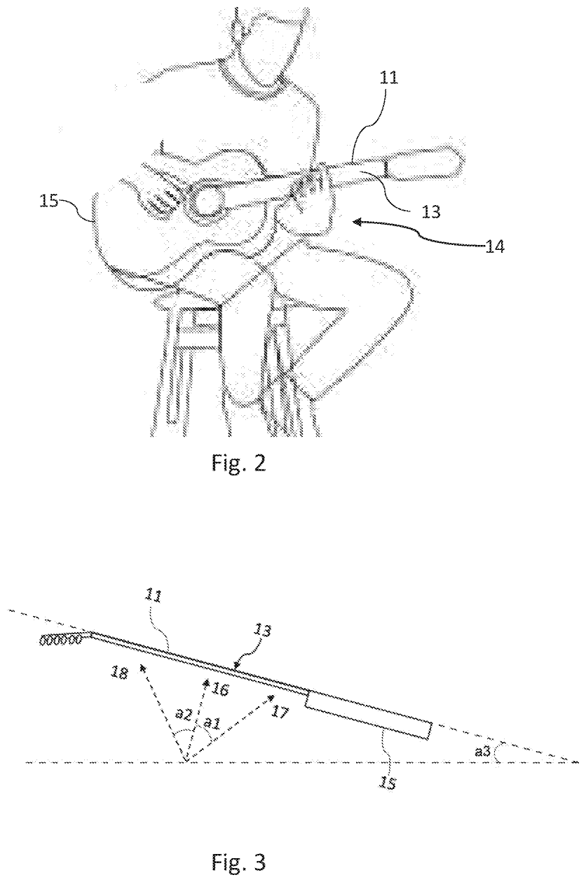

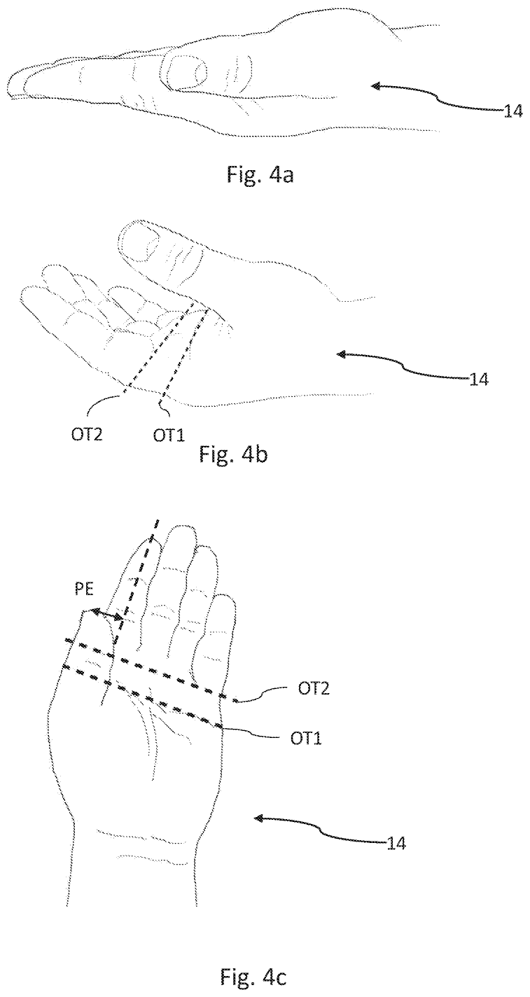

FIG. 2 shows a musician's position when playing a guitar-type stringed instrument. The musician's chord-playing hand 14 can play chords in different points of the neck 11 of the stringed instrument 10 such that the thumb of the chord-playing hand 14 settles on the back of the neck 11 and the other fingers of the chord-playing hand 14 settle on the front side of the fretboard 13 where they can press the strings 12.

FIG. 3 shows a side view of the stringed instrument which is in the typical playing position seen from the musician's top profile in a specific “forward-backward” angle a3 in relation to the musician's anatomical frontal axis, and positions of the forearm in relation of the neck of the stringed instrument 10 from one position of the elbow joint of the chord-playing hand 14. Seen from the top profile, the musician's forearm in the internal rotation is set substantially at a straight angle in relation to the neck 11 and the fretboard 13 of the stringed instrument, which is illustrated by a dashed arrow 16. When the musician plays the chord from a high register 17 closer to the sound box or body 15 of the stringed instrument 10, the forearm 14 of the chord-playing hand settles at an angle which is here designated by a1. Equivalently when the musician plays the chord from a low register 18 of the neck 11 at the end opposite to the body, the forearm of the chord-playing hand 14 settles at an angle which is here designated by a2. The wrist of the musician's chord-playing hand 14 is in the zero position substantially parallel with the forearm, but it can also settle in flexion, whereby the palm's angle in relation to the forearm is here b1, or the wrist can settle in extension, whereby the palm's angle in related to the forearm is here b2. The internal rotation/external rotation of the forearm towards the neck is primarily 0 degrees, when the forearm has been set by means of it to a substantially perpendicular 16 relation with the instrument's neck 11 and fretboard 13.

Those skilled in the art will find it obvious that, as technology advances, the basic idea of the invention can be implemented in many different ways. The invention and its embodiments are thus not restricted to the described examples but may vary within the scope of the claims.

It is characteristic of a neck of a stringed instrument shaped by means of the method that, in its neck profile, on the opposite side of its fretboard is identified a kind of a guide run for the thumb, which corresponds to a route along which the musician prefers to shift their thumb when moving their chord-playing hand on the neck forward or backward in the direction of the neck. This guide run does not run symmetrically at a specific height degree but moves on the surface of neck from one height point to another, simultaneously being of uniform feel to the thumb. A transverse location of said guide run of the neck and, simultaneously, a maximum thickness of the neck in different points of its longitudinal direction can be determined by measuring the hand from the hand's natural movement limits. The dimensioning and profile of the neck of a stringed instrument can be decided based on the determination of the guide run.

The amount of the wrist's flexion is developed as the musician tries to maintain a smooth and natural feel to the guide. When doing this, the musician's chord-playing hand's upper limb joints are guided to positions that support playing without the musician needing to concentrate on it. By means of the guide run, the chord-playing fingers are also guided on the side of the fretboard starting from the low register of the fretboard perpendicularly to about a position being in front of the forearm such that the extend of the chord of the fingers in the neck direction corresponds to as well as possible in each position a distance of frets corresponding to the front finger and the little finger. When synchronising the chord-playing fingers with the fret distances, it is naturally possible to focus on synchronising any combination of the chord-playing fingers. In the neck shaped this way, the thumb, chord-playing fingers and hand positions are guides and determined to a great extent in a passive way and as if automatically as the musician focuses on the good feel. Potentially, this will make playing considerably more relaxed, accurate and fast.

Next will be introduced required terminology to describe the details of the invention.

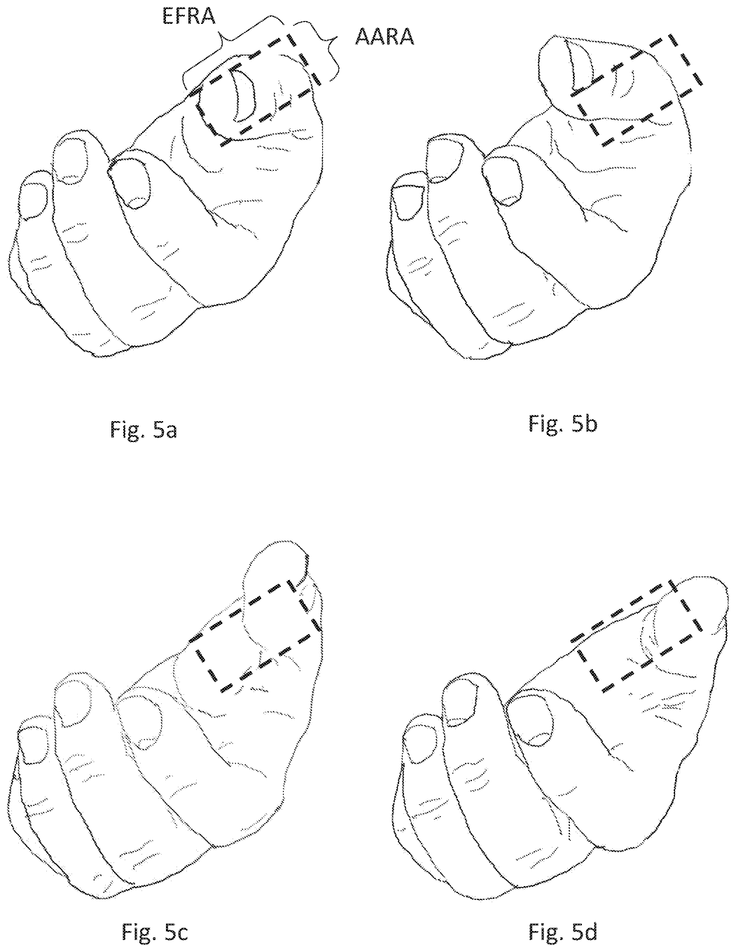

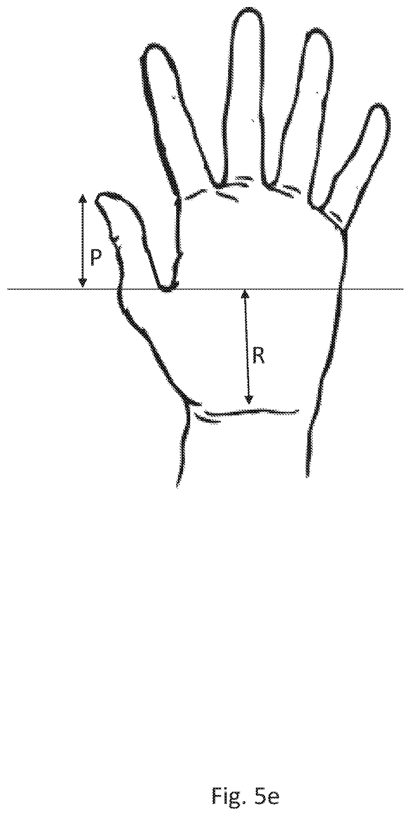

Relaxed point after extension (EJRP) is a term which is used in describing a relaxed state to which a joint passively returns from such an extension state which, without assistance, requires active muscle work to remain in the state in question. EJRP is reached by letting the muscles maintaining the extension state to relax and by letting the joint to return to the relaxed state without active muscle work or intention to flexion. This is shown in FIG. 5c.

Relaxed point after flexion (FJRP) is a term which is used in describing a relaxed state to which a joint passively returns from such a flexion state which, without assistance, requires active muscle work to remain in the state in question. FJRP is reached by letting the muscles maintaining the flexion state to relax and by letting the joint and the fingers to return to the relaxed state without active muscle work or intention to extension. This is shown in FIG. 5a.

Relaxed point after abduction (AbJRP) is a term which is used in describing a relaxed state to which a joint passively returns from such an abduction state which, without assistance, requires active muscle work to remain in the state in question. AbJRP is reached by letting the muscles maintaining the abduction state to relax and by letting the joint and the fingers to return to the relaxed state without active muscle work or intention to adduction. This is shown in FIG. 5b.

Relaxed point after adduction (AdJRP) is a term which is used in describing a relaxed state to which a joint passively returns from such an adduction state which, without assistance, requires active muscle work to remain in the state in question. AdJRP is reached by letting the muscles maintaining the adduction state to relax and by letting the joint and the fingers to return to the relaxed state without active muscle work or intention to abduction. This is shown in FIG. 5d.

Relaxed area of extension-flexion (EFRA) includes an area between EJRP and FJRP, within which positions reached by these movements do not require active muscle work for maintaining them. Exceeding limit values refers to active muscle work in order to maintain the state. This is denoted in FIG. 5a.

Relaxed area of abduction-adduction (AARA) includes an area between AbJRP and AdJRP, within which positions reached by these movements do not require active muscle work for maintaining them. Exceeding limit values refers to active muscle work in order to maintain the state. This is denoted in FIG. 5a.

The movements of the thumb's joints are complex. For the clarity of the measuring method, the movements flexion/extension and abduction/adduction starting from the thumb's carpometacarpal (CMC) joint are easiest to perceive starting from the zero position of the hand, wherein:
flexion/extension moves the thumb around its base on a plane parallel with the plane of the palm, like a clock-hand against the clock face
abduction/adduction moves the thumb around its base on a plane perpendicular with the plane of the palm.

In the method for forming the neck 11 of the stringed instrument 10, the following measurements are made according to an embodiment:

### Thumb Measurements

measuring the musician's palm length R in the direction of the forearm from the wrist's folding point until the crease corresponding to the base of the thumb;
measuring the musician's thumb length P from the crease of the ulnar base until the tip of the thumb;
measuring the distance of the musician's thumb tip PE perpendicularly to the tip of the front finger along an imaginary line running substantially parallel with the forearm;
measuring the relaxed point after the combined flexion-abduction movement of the thumb and an angle of abduction and an angle of flexion/extension in accordance with it;
measuring the relaxed point after the combined extension-abduction movement of the thumb and an angle of abduction and an angle of flexion/extension in accordance with it;

### Settling of Fretboard on Palm Plane

calculating the range of variation of the edge next to the palm of the plane of the fretboard (13) of the neck (11) of the stringed instrument (10) rising from the plane of the palm straight forward-backward on the palm's plane by calculating the distance between OT2 and OT1 aligned together;
calculating the range of variation of the angle next to the palm of the plane of the fretboard (13) of the neck (11) of the stringed instrument (10) rising from the plane of the palm between OT1 and OT2 into both directions, an imaginary straight line crossing on one side with OT1 and the other side with OT2 of the plane of the palm

When both the thumb length and its relaxed points are known, it is possible to deduce the thumb's point on the neck in relation to the chord-playing finger. Of the relaxed points, the relaxed points after the combined flexion-abduction movement and the combined extension-abduction movement are important when deducing the maximum thickness of the neck at the point of the tip of the thumb being perpendicular to the plane of the fretboard. These are shown in FIGS. 5a-5d. Between these two relaxed points, there can be imagined to be a straight line where the thumb's flexion-extension moves and offers a range of variation where it can increase or decrease an angle between the thumb and the chord-playing finger by increasing or decreasing the space reserved for the instrument's neck and the distance from the plane of the fretboard to the point of the thumb. The relaxed points after the combined extension-adduction movement and the combined flexion-adduction movement again show an adjustment allowance by which the thumb intentionally approaches the fretboard 13 and the neck. By means of them, it is also possible to deduce the minimum thickness of the neck at the tip of the thumb being perpendicular from the plane of the fretboard. The maximum thickness of the neck 11 of the stringed instrument 10 is calculated at the point of the thumb being perpendicular to the fretboard by deducting the length of the thumb from the distance between the base of the thumb and the bases of the chord-playing fingers on the palm in an angle in accordance with the combined flexion-abduction movement of the thumb.

### Natural Longitudinal Reach of Chord-Playing Fingers

The position is formed for a stringed instrument of four successive fret distances and each of the four chord-playing fingers represent one fret distance in the position. Such an arrangement is commonly called “one finger per fret distance”.

The natural reach of relaxed chord-playing fingers in the longitudinal direction of the neck typically changes in the flexion and extension of the wrist. This information can be effectively utilised in the shaping of the neck profile when deciding on the degree of the wrist's flexion in those positions and points where the wrist's flexion is used. The purpose of the correct wrist flexion is the positioning of both the front finger and the little finger as well as possible in the “finger per fret distance” arrangement to the immediate vicinity of the frets corresponding to them. This enables ergonomic muscle work when the fingers are set on the strings. The aim is thus to match the natural reach of the chord-playing fingers as well as possible by the specific amount of the wrist's flexion with the distance of four frets in the specific position.

The degree of the wrist's flexion essentially affects the position of the thumb on the neck of the instrument, especially the perpendicular height point on the side of the neck calculated from the edge corresponding to the plane of the fretboard, and it is best to form the thickest point in the neck in this point of the thumb in the point in the longitudinal direction of the neck in question.

### Determination of Wrist Flexion

The amount of the wrist's flexion to best serve each position can be selected by testing the natural reach of the chord-playing fingers in different degrees of flexion. It is also easy to form a simple calculation formula to support the selection.

The distance between four successive frets in the position has an effect on which degree of wrist flexion is selected in the position in question when determining the thickest point of the neck. It is wished that the natural reach of the fingers matches the reach of the frets as well as possible. The amount of flexion is then at its greatest in the first position and decreases until the position corresponding to the zero position is reached. The hand is not made to turn on the side of extension when determining the thickest point. In a stringed instrument, the position where the wrist's flexion reaches 0 degrees varies depending of the musician's anatomy and physique, playing position and instrument's angles. By default, it is found between positions 5-9.

### Calculation of Wrist Flexion

In the wrist's zero position, abduction of the chord-playing fingers is performed and they are let to relax and settle passively in their own position. The distance between the tips (centre points) of the forefinger and the little finger is measured and it is recorded as the reach of the chord-playing fingers after abduction in the wrist's zero position.
The hand is positioned vertical from the forearm and the wrist is let to relax totally in the direction of the wrist's flexion. The degree of the wrist's flexion is measured, the wrist being passive in its maximal flexion, and it is recorded as the wrist's maximal flexion.
The wrist being passive in its maximal flexion, abduction of the chord-playing fingers is performed and they are let to relax and settle passively in their own position. The distance between the tips (centre points) of the forefinger and the little finger is measured and it is recorded as the reach of the chord after the abduction of the chord-playing fingers in the wrist's maximal flexion.
The change in the width of the chord of the chord-playing fingers between the wrist's zero position and maximal flexion is calculated. The number corresponding to the change is divided by the degree reading of the wrist's maximal flexion, whereby the effect of one degree in the change in the natural reach of the chord-playing fingers after the abduction is deduced.
When calculating the reach of the chord of the chord-playing fingers in the longitudinal direction of the neck, the change reading corresponding to one degree in the wrist's flexion is multiplied by the reading of the wrist's flexion and it is added to the reach of the chord-playing fingers after the abduction in the wrist's zero position.
Using the same logic, it is possible to measure the range of variation of the natural reach of the chord in the longitudinal direction of the neck after the adduction of the chord-playing fingers between the wrist's zero position and maximal flexion, and calculate the effect of one degree in the change in the natural reach of the chord-playing fingers and the reach of the chord based on the arrangement corresponding to each situation.

The formulas formed based on both the abduction and the adduction of the chord-playing fingers are useful. Depending on the musician's anatomy, physique and position, it is possible to decide to use either one of the formulas when determining the wrist's flexion, or both of them, whereby the thumb is determined with at least two different locations and maximum thicknesses determined for them. A suitable rounding or plane of the profile can be formed between these points.

By means of this, it is possible to calculate the effect of the amount of the wrist's flexion in the natural reach of the chord-playing fingers in the longitudinal direction of the neck and to try to match the reach of the chord-playing fingers with a specific amount of flexion as accurately as possible for each position.

### Described by Formulas:

$$Natural reach of chord - playing fingers in longitudinal direction of neck after abduction = reach of chord after abduction of chord - playing fingers in wrist ’ s zero postion + ( ( wrist ’ s flexion angle ) \times ( effect of one degree in change in natural reach of chord - playing fingers after abduction ) )$$

$$Natural reach of chord - playing fingers in longitudinal direction of neck after abduction = reach of chord after abduction of chord - playing fingers in wrist ’ s zero postion + ( ( wrist ’ s flexion angle ) \times ( effect of one degree in change in natural reach of chord - playing fingers after abduction ) )$$

$$Natural reach of chord - playing fingers in longitudinal direction of neck matched with measured postion = natural reach of chord - playing fingers in longitudinal direction of neck \approx distance between frets corresponding to little finger and forefinger$$

### Range of Variation of Fretboard Plane and Chord-Playing Fingers' Depth Points

measuring the distance in the direction of the forearm from the base of the thumb to an imaginary straight line (OT1) describing the creases of the base joints;
measuring in the direction of the forearm the shortest distance from the imaginary straight line (OT1) describing the creases of the base joints of the chord-playing fingers to another imaginary straight line (OT2) to the creases of the fingers' base members, where the fingers start to visibly separate from each other outside the rest of the palm;
measuring the normalised width of the palm from the range of variation between OT1 and OT2;
OT1=proximal limit of fretboard plane in the palm
OT2=distal limit of fretboard plane in the palm

OT1 and OT2 provide a range of variation where the edge corresponding to the plane of the fretboard of the neck settles or is projected on the plane of the palm in different positions of the hand in various points of the neck. Adjustment caused by the angles of the joints of the upper limb forward and backward within the range of variation can occur on either side of the palm (on the side of the forefinger or the little finger) or on both sides—in the same or in a different ratio. If the edge corresponding to the plane of the fretboard of the neck settles or is projected at an angle with the line in the OT1 and OT2 direction normalised on the plane of the palm, this affects the ratios of between the thumb and the chord-playing fingers substantially and will be thus considered as the “forward-backward” offset of the chord-playing hand when determining the profile, inter alia, in the angle of the thumb toward the neck.

When the edge corresponding to the plane of the fretboard of the neck settles or is projected on the plane of the palm at any angle within said range of variation, the tips of the chord-playing fingers are often settable to a linear playing position in relation to each other in a passive and relaxed state almost at the same distance from the string and the plane of the fretboard. By means of the chord-playing fingers, it thus often possible to compensate said “forward-backward” offset of the chord-playing hand by bringing the chord-playing fingers otherwise ending up farther away from the fretboard in the depth direction closer to the fretboard settling between OT1 and OT2 in the palm.

### Described by Formulas:

$$Primary range of variation in plane of fretboard on plane of palm = OT 2 - OT 1$$

$$Primary range of variation in angle of fretboard on plane of palm in depth direction = a tan ( range of variation / palm ’ s normalised width in range of variation ) * 180 / π$$

According to an embodiment of the method, the height points of the chord-playing fingers and the effect of the adjustment range of the linear position are measured, that is, the effect of the various positions of the wrist, on the dimensioning.

### Height Points of Chord-Playing Fingers and Adjustment Range of Linear Position

Measuring the highest height point (OT1S2) of the finger which extends to the shortest from the palm plane OT1 when the wrist is straight
Measuring the highest height point (OT1F2) of the finger which extends to the shortest from the palm plane OT1 when the wrist is in its maximal natural flexion
Measuring the highest height point (OT2S2) of the finger which extends to the shortest from the palm plane OT2 when the wrist is straight
Measuring the highest height point (OT2F2) of the finger which extends to the shortest from the palm plane OT2 when the wrist is in its maximal natural flexion

The wrist's flexion extends the fingers away from the palm thus lifting their height points. By means of the straight wrist and the flexed wrist, it is possible to make a linear algorithm from the effect of the flexion of the wrist on the height points of the linearly positioned fingers from the palm on the plane of OT1 and OT2. The different tilting angles of the wrist are used in a varied way and the effect of this on the position of the chord-playing fingers and the thumb is important when shaping the profile.

The highest common height point of the linearly settable chord-playing fingers on the fretboard is determined based on where, in the playing position, the highest height point settable on the string of the finger tip extending shortest to the edge corresponding to the plane of the fretboard of the neck perpendicular in relation to the neck and strings extends. The lowest common height point of the linearly settable chord-playing fingers on the fretboard is determined based on where, in the playing position, the lowest height point settable on the string of the finger tip extending longest to the edge corresponding to the plane of the fretboard of the neck perpendicular in relation to the neck and strings extends when the finger advances the string and the fretboard substantially perpendicularly. The lowest and the highest height point of the linear position of the chord-playing fingers on the fretboard provide a range of variation which is dependent on in each point of the neck from the positions of the joints of the upper limb, which again guide the thumb to a specific point on the back of the neck. This information can be used by measuring the position of the thumb in accordance with two or more positions such that the range of variation of the chord-playing fingers can be made to cover a larger area when the thumb moves in a relaxed way on the surface of the neck from one point to another along with the adjustment of the angles of the other joints. The larger the flexion in which the hand comes onto the neck, the higher the range of variation of the linearly set chord-playing fingers on the side of the fretboard naturally is.

### Described by Formulas:

range of variation on OT1 plane with straight wrist: OT1S2-OT1S1
range of variation on OT1 plane in wrist's maximal flexion: OTF2-OTF1
range of variation on OT2 plane with straight wrist: OT2S2-OT2S1
range of variation on OT2 plane in wrist's maximal flexion: OT2F2-OT2F1

### Shaping of Profile

Depending of the musician's anatomy and physique, the thickest points of the neck are in different points in the instrument's neck. Around the thickest points of the neck, a profile can be shaped in various ways.

In the shaping of the neck profile, the initial thickness of the thickest point in the neck in the first position is selected within the range allowed by the relaxed points. The necks often get thicker linearly towards the high register, or they can also stay the same or even become thinner. The thickest point of different positions equals the values allowed by the relaxed points in the end result. As an example, the initial value and the end value of a linearly thickening neck is decided from within the values allowed by the measurements and the thickest thickness of the neck is then obtained in each position in accordance with the thumb's longitudinal position. The height point of the thickest point in the neck is largely determined by the amount of the wrist's flexion in those position where the wrist flexion is used.

At the thickest point of the neck, it is possible to shape a smoother area of its feel for the thumb in the profile, which helps to guide the thumb on the back of the neck. When determining the thickest point of the neck profile, the wrist is not intended to move on the side of extension over the zero position.

When the forearm turns toward the saddle (i.e. the high register), at the latest in the stage when the palm's OT2 is on the thumb side further than the lower edge of the plane of the fretboard of the neck on the side of the little finger, the thumb starts to need more space toward the fretboard and then the profile must be “cut” in accordance with the thumb's coordinates. The thumb is provided more space by moving the thumb from the relaxed point of FIG. 5b to the relaxed point of FIG. 5c or gradually toward it. At the risk of too radical a cut, it is case-specifically possible to allow the thumb to have a little moving/additional space over the relaxed point.

The purpose is the shape the profile into a unit working in the instrument. The profile can be determined more freely for the part what different approaches do not determine of the profile.

The thickest point in the neck in each longitudinal point already determines the shape of the neck largely. If wishing to avoid sudden or “hilly” changes of the thickest point in the longitudinal direction of the neck, the neck can be for its whole length or partially from its thickest point be either unchanged or linearly changing. For a linearly thickening or thinning neck, the initial thickness and the end thickness as well as the corresponding to longitudinal points in the neck are decided. The end result must still follow the limit values specified by EFRA and AARA, whether the thickness is unchanged or linearly changing or asymmetrically varying.

The shaping of the rest of the neck also largely utilises the thumb's EFRA and AARA relaxed areas such that the thumb can slide relaxedly into another point on the surface of a neck shaped based on them, even though the other angles of the joints of the upper member of the chord-playing hand remain unchanged and the chord-playing hand remains in its place. The thumb can move above and/or below the thickest point of each longitudinal point from the thickest point of another longitudinal point following the rounding of the neck shaped by means of the EFRA and AARA relaxed areas.

Several coordinates can be specified by the wrist's flexion-extension in the position, by means of which, the profile can be e.g. rounded above and/or below the thickest point. When shaping the total thickness of the neck, the thickness and height of the fretboard from the plane of the fretboard to the surface of the strings have to be considered.

### Described by Formulas:

$$Angle corresponding to thickening of linearly thickening neck = A TAN ( change in neck ’ s thickness for thickening distance \div total length of thickening distance in direction of neck ) \times 180 \div π$$

$$Thickness of thickest point in linearly thickening neck in thumb ’ s longitudinal point = initial thickness of neck ’ s linearly thickening part + length of thickening distance in direction of neck from initial point to point of thumb \times SIN ( angle corresponding to thickening of linearly thickening neck \times π \div 180 )$$

$$Neck ’ s maximum thickness at the point of thumb other angle coefficients = ( OT 1 + OT 2 ) - ( P \times cos ( thumb ’ s extension degree \times π \div 180 ) \times cos ( thumb ’ s abduction degree \times π \div 180 ) )$$

$$Neck ’ s maximum thickness at the point of the thumb considering the angles of the joints : ( ( OT 1 + OT 2 ) \times COS ( total angle of incidence of wrist ’ s flexion / extension \times π \div 180 ) ) - ( P \times COS ( ( flexion / abduction degree after combined flexion - abduction movement + total angle of incidence of wrist ’ s deviation ) \times π \div 180 ) \times COS ( ( total angle of incidence after combined flexion - abduction movement + wrist ’ s flexion / extension ) degree \times π \div 180 ) )$$

$$Total angle of incidence of wrist ’ s flexion / extension = wrist ’ s flexion / exension + forearm ’ s flexion / extension + upper arm ’ s flexion / extension$$

$$Total angle of incidence of wrist ’ s deviation = wrist ’ s ulnar / radial deviation + upper arm ’ s internal rotation / external rotation$$

The movements of the chord-playing hand from the shoulder joint to the wrist in relation to the instrument's neck can also affect the dimensioning.

When playing, the neck of the stringed instrument is rarely horizontal or parallel with the musician's horizontal plane seen from the front. The pronation/supination movements of the forearm and, if necessary, also the abduction/adduction movements of the upper arm can be used to match the palm plane parallel to the neck's angle. If the palm's plane is not substantially at the same angle with the angle rising upward (or lowering downward) of the neck, it is considered to offset “upward-downward” from the angle of the neck”, which has an effect on the positioning of the chord-playing fingers and the thumb on the fretboard and the neck.

When playing, the side profile of the neck of the stringed instrument is rarely parallel with the musician's frontal axis seen from the front, in relation which, it is often tilted from the side of the headstock forward from the musician. Such a “forward-backward” angle 13 can be compensated in a relaxed way by the upper shoulder's internal flexion (or external flexion if necessary) in order to make the forearm to advance the neck 11 and the fretboard 13 of the stringed instrument without otherwise changing the playing position.

The wrist's ulnar deviation/radial deviation, the forearm's internal rotation/external rotation and the upper shoulder's abduction/adduction movements substantially affect the positioning of the thumb and the hand in relation to the neck, and they are used to match the palm's OT2 or OT1 line parallel with the lower edge of the plane of the fretboard. In some positions, the lower edge of the plane of the fretboard can settle at an angle within the limits allowed by the range of variation, and this affects the thumb's position on the neck.

Changes in angle toward fretboard 13 of the stringed instrument in the longitudinal direction of neck 11:
by upper shoulder's internal rotation or external rotation (and upper shoulder's abduction or adduction possibly combined to it) and, to a lesser extent, the wrist's ulnar and radial deviation, when the fretboard/neck is thought to be horizontal and the forearm is in supination in the zero position of the playing of the stringed instrument in order to be able to position the thumb and the chord-playing fingers correctly on the neck and the fretboard
by elbow joint's extension and flexion and, to a lesser extent, the wrist's ulnar and radial deviation, when the fretboard/neck is thought to be vertical and the forearm is in pronation of 90 degree compared with the zero position of the playing of the stringed instrument in order to be able to position the thumb and the chord-playing fingers correctly on the neck and the fretboard

Changes in angle toward fretboard 13 of the stringed instrument in the width direction of neck 11:
by shoulder joint's flexion or extension, elbow joint's flexion or extension, the wrist's flexion or extension, when the fretboard/neck is thought to be horizontal and the forearm is in supination in the zero position of the playing of the stringed instrument in order to be able to position the thumb and the chord-playing fingers correctly on the neck and the fretboard
by upper shoulder's internal rotation or external rotation (and upper shoulder's abduction or adduction possibly combined to it) and the wrist's flexion or extension, when the fretboard/neck is thought to be vertical and the forearm is in pronation of 90 degrees compared with the zero position of the playing of the stringed instrument in order to be able to position the thumb and the chord-playing fingers correctly on the neck and the fretboard

The dimensions can be measured mechanically by using various tools, such as rulers and angle measurers. The points used in the measurements can be marked by a pen or some other tool on the upper limb and, thus, facilitate the perception of the points being measured.

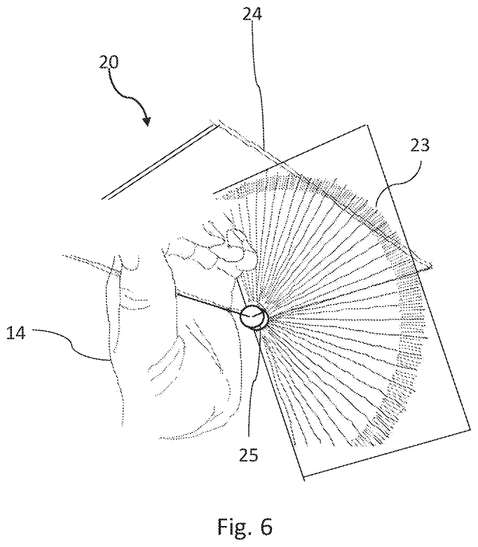

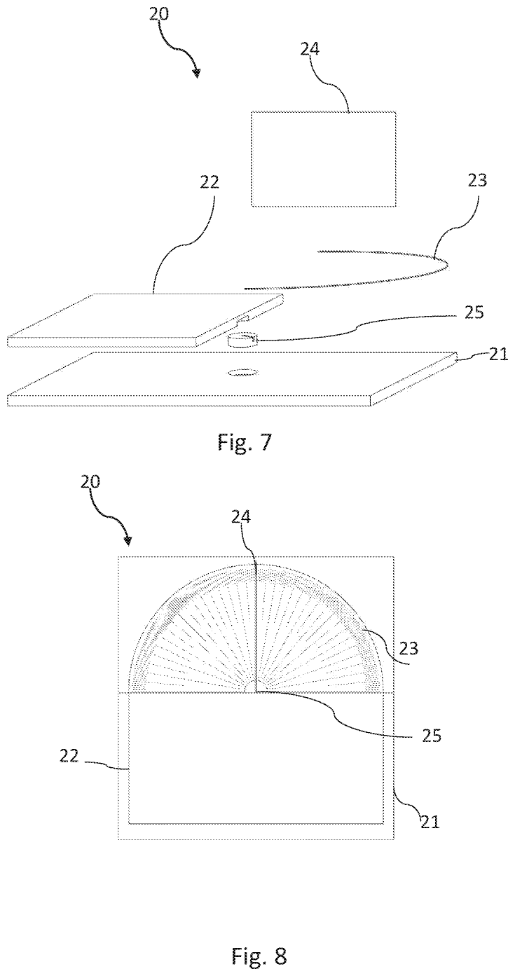

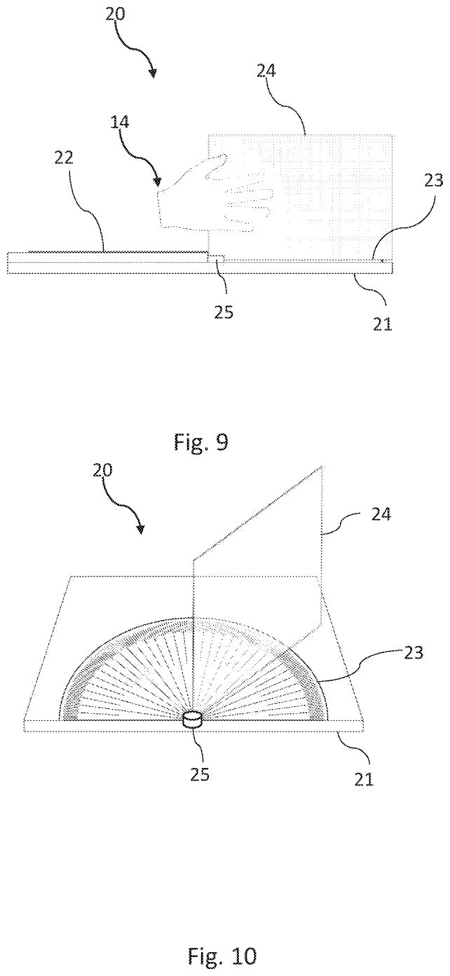

According to an embodiment of the method, a profile measurer 20, which is shown in FIGS. 6-10, is utilised in performing the measuring. The profile measurer comprises a base plate 21, a hand support 22, a scale plate 23, a measuring plate 24, which is transparent and manufactured of e.g. glass or plastic. The measuring plate 24 is hinged to the scale plate 23 by means of a hinge 25. The transparent measuring plate 24 enables the reading of accurate measurements from the other side of the touch surface. The measuring plate 24, is rotated on top of the scale plate 23 around the axis substantially for 180 degrees. The scale plate 23 has degree marks, from which, it is possible to read the degree between two items being measured by rotating the measuring plate 24. Such items in the profile measurements of the stringed instrument 10 are, inter alia, the thumb kept on the back of the neck 11 and the chord-playing fingers on the side of the fretboard 13. When performing degree measurements, the palm of the chord-playing hand 14 is set on the measuring plate 24 on the edge on the side of the hinge 25 such that the thumb is on the other side of the measuring plate 24 and the other fingers on the opposite side. The palmar side of the chord-playing fingers is toward the measuring plate 24 and their dorsal side away from the measuring plate 24. The forearm is in pronation such that the palm's plane can be set on the edge on the side of the hinge 25 of the measuring plate 24. The forearm's pronation has no substantial effect on the measurement results. The wrist's flexion and extension have an effect on the measurement results because it changes the positions of the chord-playing fingers' joints. The thumb turns in the same ratio with the wrist. When measuring by the profile measurer 20, the forearm's extension and flexion can simulate the changes in angles occurring in the upper arm's internal rotation and external rotation in relation to the neck 11 of the stringed instrument 10. The upper arm' internal rotation and external rotation can again simulate the changes in angles occurring in the forearm's flexion and extension in relation to the neck 11 of the stringed instrument 10. By adjusting these, it is possible to simulate the position of the chord-playing hand 14 on the neck 11. The points used in the measurements can be marked by a pen or some other tool on the upper limb and, thus, facilitate the distinction of the items being measured through the measuring plate 24.

The measurement values, based on which the degree between the thumb and any one of the chord-playing fingers is determined, are read from the scale plate 22 at points where the measuring plate 23, when rotated, hits the fingers being measured in a gentle manner, without moving them. The measuring plate 23 is accurately installed in a centralised way on top of the degree marks and the side of the measuring plate 23 hitting the finger changes between the thumb and the chord-playing fingers, whereby the thickness of the measuring plate 23 can be considered in the measurement results.

The measuring plate 23 includes millimetre rules to read the distance between the fingers and the thumb measured from the edge on the side of the hinge 24 of the measuring plate 23. They are marked in accordance with the separate measuring instructions. By means of the millimetre rules, the distance of the fingers is also read, especially the distance of the thumb from the forefinger and/or middle finger. The distance is read in the direction of the edge on the side of the hinge 24 of the measuring plate 23. The transparent measuring plate 23 also enables the measurement of other things, such as the width and height of the fingers' contact surface. The contact surface refers to the finger's area which presses fast on the surface when touching it lightly. This dimension also has its own value, for example when deducting the suitable distance of the strings 12 in the construction of the stringed instrument. By means of the distances of the chord-playing fingers, it is also possible to deduct the suitable scale length of the neck 11, that is, the vibrating length of the strings that produce sound.

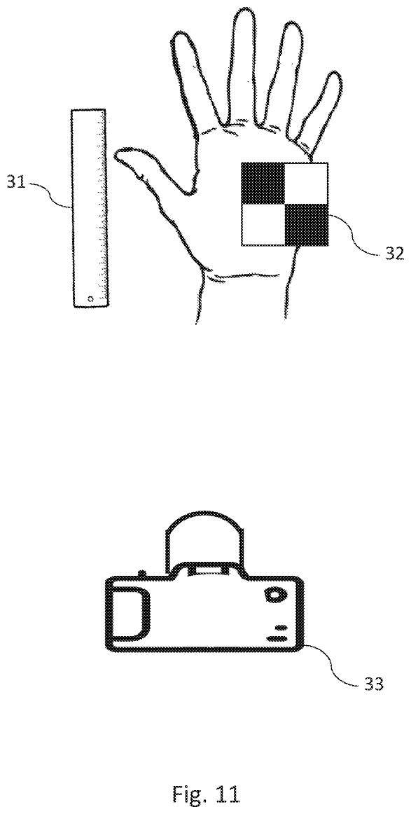

According to an embodiment of the method, the measurements are performed by shooting images such that one marker or more i.e. a reference measure has been put beside the chord-playing hand 14 substantially at the same distance from the camera 33. This is shown in FIG. 11. By means of the reference measure 30, the scale of the image can be ensured and distortions due to the imaging angle can be fixed. The reference measure 30 can also be specified and combined to the image digitally such that the accurate distance of the target being imaged and the focal length of the camera lens are known. The reference measure 30 can be a measuring scale 31, by means of which, the actual dimension of the imaging target is calculated from the image according to the scale of the measuring scale 31. The reference measure 30 can also be a graphic two-dimensional pattern 32, of which, it is possible to identify an image part or parts of standard dimensions and standard shapes, e.g. a square or squares. The graphic pattern 32 is located substantially on the same plane as the measuring target, such as e.g. the thumb or the palm. An angle between the camera and the imaging target is calculated from the graphic two-dimensional pattern. If the graphic pattern is visible in the image as a standard shape, like said square, it is possible to assess that the imaging target is substantially at a right angle in relation to the camera and no significant measurement error has been created. If the graphic pattern 32 in the image is distorted from its standard shape, e.g. the square shows as a parallelogram or an asymmetric quadrangle, a correction coefficient of the quantity of the distortion is calculated to the measurement results calculated from the image. The images can be shot e.g. by a camera, scanner or web camera. When imaging by the scanner, the chord-playing hand 14 and the reference measure 30 are set on the imaging plate/imaging table of the scanner. Ideally, the reference measure 30 covers the item being imaged in the horizontal and vertical direction. When imaging, it is also possible to effectively utilise the transparent plane against which the item being measured is set and which includes the reference measure 30 or several of them either fixedly or fastened, like in the measuring plate of the profile measurer 20. The points used in the measurements can be marked by a pen or some other tool on the upper limb and, thus, facilitate the visibility of the items being measured.

### Formulas Supporting the Method

The formulas based on the measuring method can refer to different angles of the upper limb's joints as pairs of opposites. These are the upper arm's abduction/adduction, flexion/extension and internal rotation/external rotation, the forearm's flexion/extension and pronation/supination, the wrist's flexion/extension and ulnar deviation/radial deviation, and the thumb's abduction/adduction and flexion/extension. For all of these, one is referred to by a positive number and the other is referred to a negative number. As an example, the forearm's flexion/extension in the formula refers to the forearm's flexion if the angle is a positive number, and to extension if the angle is a negative number.

The upper arm's adduction, flexion and internal rotation are designated by positive numbers, and their opposites, in the corresponding to order, abduction, extension and external rotation are designated by negative numbers. The forearm's flexion and pronation are designated by positive numbers, and their opposites, in the corresponding to order, extension and supination are designated by negative numbers. The wrist's flexion and ulnar deviation are designated by positive numbers, and their opposites, in the corresponding to order, extension and radial deviation are designated by negative numbers. The thumb's abduction and flexion are designated by positive numbers, and their opposites, in the corresponding to order, adduction and extension are designated by negative numbers.

It is typical for different playing positions that the stringed instrument and its neck are at a specific angle in relation to the musician's one or more planes and/or axes.

The upper arm's internal rotation/external rotation is interpreted as 0 degrees in relation to the neck of the stringed instrument when, by means of it, the forearm is matched to approach the neck of the stringed instrument substantially perpendicularly seen from the musician's top profile. The forearm's pronation/supination is 0 degrees toward the neck when, by means of it, the palm's plane has been matched parallel with the angle of the neck of the stringed instrument rising upward (or lowering downward).

When playing the stringed instrument, the side profile of the neck of the stringed instrument is rarely parallel with the musician's frontal axis seen from the front, in relation which, it is often tilted from the side of the headstock forward from the musician. Such a “forward-backward” angle 13 can be compensated in a relaxed way by the upper shoulder's internal flexion (or external flexion if necessary) in order to make the forearm to advance the neck and the fretboard of the stringed instrument without otherwise changing the playing position. In the formulas, the internal rotation/external rotation of the forearm towards the neck is primarily 0 degrees, when the forearm has been set to a substantially vertical 16 relation with the instrument's neck 11 and fretboard 13.

Seen from the front, the neck of the stringed instrument and, along with it, the lower edge of the neck can be substantially tilted in relation to the musician's horizontal plane, usually upward from the headstock. The angle can be gentle or radical and is greatly dependent on the musician and the selected playing position. Such an “upward-downward” angle is primarily compensated by the forearm's pronation or supination in order to have the palm's plane parallel with the edge of the neck of the stringed instrument which is beside the palm.

### More Detailed Descriptions of Measurements

Next, the actual measurements will be described in more detail, including the measurements performed by the profile measurer 20 or imaging.

### From Wrist's Folding Point Crease to Thumb's Base Plane (R)

Measuring the distance from the crease of the straight wrist's folding point to the plane of the thumb's base in the direction of the forearm of the chord-playing hand 14. To facilitate measurement, it is possible to draw a line or some other mark by a pen to the crease of the wrist's folding point and the thumb's base.
By the profile measurer 20, the measurement is most successful by setting the plane of the measuring plate and the forearm parallel and by setting the crease of the wrist's folding point following its whole length on the edge on the side of the hinge of the measuring plane to the back of the measuring plate. The distance to the plane of the thumb's base is easily readable by means of the distance marks in the measuring plate from the opposite side of the measuring plate.
When measuring from an image, the distance is measured from the image such that the wrist and the chord-playing fingers are straight and the thumb can be in a small extension on the plane of the other fingers. The image is straight from the front.

### Thumb's Length (P)

Measuring by setting the end of the ruler at the base of the thumb being kept the tip joint straightened and by reading the distance from the base to the plane of the tip from the ruler following the thumb's side.
   If the thumb turns starting from the hand's zero position (at least almost) 90 degrees into the direction of the palmar abduction, the thumb's length in the position in question is measured by the ruler from the plane of the palm perpendicularly to the tip of the thumb. Then, the thumb's length precisely equals the measure of the thumb which turns in the abduction from the zero position from the plane of palm. In this position for the part of the palm's plane and the thumb, the hand resembles a letter L seen from the side profile on the thumb side. For some, the hand position in question can only be possible when assisted.
   As an alternative measurement or as support for the previous measurement, if the thumb turns from the hand's zero position (at least almost) 90 degrees in the direction of extension, the thumb's length is measured in the position in question by the ruler from the line of the thumb being in the hand's zero position in the direction of the forearm perpendicularly to the tip of the thumb being in extension on the plane of the palm. In this position for the part of the palm's side on the forefinger and the thumb, the hand resembles a letter L upside down seen from the top profile of the palm. For some, the hand position in question can only be possible when assisted.
Measuring by the profile measurer 20 by setting the palm on the measuring plate such that the thumb's base is on the edge on the side of the hinge of the measuring plate. The chord-playing fingers are on the back of the measuring plate and the thumb is its top joint naturally extended on the front side of the measuring plate pointing horizontally on the other side of the plate. In order to ensure accuracy, the thumb is kept fast in the measuring plate. By means of the marks, the thumb's length can be read from the front side of the measuring plate 24 from the edge of the plate to the point where the tip of the thumb is.
Measuring from the image the length of the thumb being naturally its tip joint extended. In the angle of the image, the plane of the thumb's back is set toward the camera 33 and it is equally far from the camera for its whole area. The thumb is in the relaxed point after the combined flexion and abduction movement and the chord-playing fingers are in a natural and passive position.

### Thumb's and Forefinger's Distance (PE)

Measuring the distance of centre lines of the forefinger and thumb of the chord-playing hand 14 in the direction of the forearm the wrist and the palm being in the zero position.
Measured by the profile measurer 20 by setting the palm following the zero position of the chord-playing hand 14 fast on the measuring plate such that the fingers are as well as possible in a perpendicular line in relation to the edge on the side of the hinge 25 of the measuring plate 24. Reading the distance of centre lines of the forefinger and thumb in the direction of the forearm by means of the measurers of the measuring plate.
Measuring from the image the distance of centre lines of the forefinger and thumb in the direction of the forearm the wrist and the palm being in the zero position.

### Degrees of Thumb's Relaxed Points

Measuring the degrees of the thumb's relaxed points after various movements in which the thumb returns after passive movements.

Measuring in the thumb's relaxed point the angle between the thumb turning around on the plane perpendicular in relation to the palm's plane and the palm's plane at the base of the thumb and recording it as the degree of abduction corresponding to the relaxed point in question.

Measuring in the thumb's relaxed point the angle between the thumb turning around on the plane parallel in relation to the palm's plane and its zero position at the base of the thumb and recording it as the degree of flexion or extension corresponding to the relaxed point in question.
Measuring by the angle measurer by setting the back of the forearm and the wrist mutually straight against a straight plane. Performing the active movement preceding the relaxed point and letting the thumb return passively to its place. Setting the angle measurer on top of the forearm and the wrist parallel with them. Adjusting the angle of the measurer and moving the measurer on top of the forearm and the wrist until the angle matches the thumb's line setting on the neck from the thumb's base to its tip following the finger's line.
Measuring by the profile measurer 20. The chord-playing fingers are straightened and the thumb's base is pushed alignedly and into a soft touch on the edge of the measuring plate by keeping the forearm and the wrist straight. The thumb and the fingers can initially keep the plate in place by small compression. Performing the active movement preceding the relaxed point and letting the thumb relax and move passively to the relaxed point. Rotating the measuring plate and reading from the degree marks of the measuring plane how many degrees the measuring plate having corresponding to the forearm's plane turns when it hits the thumb's contact surface.
Measuring from an image which has been shot from the forearm's and wrist's side profile and, in which, the positioning equals the positioning of the mechanical measurement.

### From Thumb's Base to Plane of Creases of Chord-Playing Fingers' Base Joints (OT1)

Measuring the distance in the direction of the forearm from the base of the thumb to the plane of the creases of the base joints of the chord-playing fingers.
By the profile measurer 20 by setting the palm to the back of the measuring plate such that the crease corresponding to the wrist's folding point is parallel with the edge on the side of the hinge of the measuring plate. The wrist and the chord-playing fingers are kept straight and the thumb is on the plane of the other fingers in a small-scale extension. The palm is gently fast on the surface of the measuring plate 24, from the other side of which by means of the measurers, the distance at the point of the thumb's base to the creases of the chord-playing fingers' base joints is read.
From the image, the wrist and the chord-playing fingers being straight and the thumb being on the plane of the other fingers in a small-scale extension. Alternatively, the measurement can be performed in the zero position of the chord-playing hand forward from the forearm but, then, it is good to make a mark in the palm on the plane of the thumb's base joint. The image is straight from the front of the palm.

### From Thumb's Base to Plane of Creases of Chord-Playing Fingers' Base Joints (OT2)

Measuring in the direction of the forearm the distance from the plane (OT1) of the creases of the base joints of the chord-playing fingers to the creases of the base members of the fingers, where the fingers start to visibly separate from each other outside the rest of the palm (OT2); normalising the plane between the forefinger's and little finger's creases in question.
By the profile measurer 20 by setting the palm to the back of the measuring plate such that the crease corresponding to the wrist's folding point is parallel with the edge on the side of the hinge of the measuring plate. The wrist and the chord-playing fingers are kept straight and the thumb is on the plane of the other fingers fast on the surface of the measuring plate. Reading from the other side of the measuring plate 24 the distance from the creases of the chord-playing fingers' base joints (OT1) to the normalised plane where the fingers separate from the palm (OT2).
Measuring from the image, the wrist and the chord-playing fingers being straight and the thumb being set on the plane of the other fingers. Image straight from the top of the palm as if shot by a scanner.

$$Primary range of variation in plane of fretboard 13 on plane of palm = OT 2 - OT 1$$

$$Primary range of variation in angle of fretboard 13 on plane of palm in depth direction = a tan ( range of variation / palm ’ s width ) * 180 / π$$

The palm's width at the points of OT2 and OT1 enables the detection of the angles at which the fretboard 13 can settle within the limits of the range of variation. The palm's width being 10 cm and the range of variation being 2 cm, it is possible to calculate the maximum angle of the fretboard in the depth direction in relation to the palm with the formula:

$$a tan ( range of variation / palm ’ s width ) * 180 / π$$

Chord-Playing Fingers' Height Points and their Range of Variation
Measuring, the wrist being straight, the height point of the finger extending farthest from the palm's plane OT1 from the point where the chord-playing finger comes perpendicularly to the plane in question, or as close to perpendicular as possible without exceeding the limit of the perpendicular finger onto the side of the palm (OT1S1).
Measuring, the wrist being in its maximum natural flexion, the height point of the finger extending farthest from the palm's plane OT1 from the point where the chord-playing finger comes perpendicularly to the plane in question, or as close to perpendicular as possible without exceeding the limit of the perpendicular finger onto the side of the palm (OT1F1).
Measuring, the wrist being straight, the height point of the finger extending farthest from the palm's plane OT2 from the point where the chord-playing finger comes perpendicularly to the plane in question, or as close to perpendicular as possible without exceeding the limit of the perpendicular finger onto the side of the palm (OT2S11.
Measuring, the wrist being in its maximum natural flexion, the height point of the finger extending farthest from the palm's plane OT2 from the point where the chord-playing finger comes perpendicularly to the plane in question, or as close to perpendicular as possible without exceeding the limit of the perpendicular finger onto the side of the palm (OT2F1).
Measuring by using e.g. the ruler by setting the ruler as if a plane of the fretboard which rises from the palm's plane from the crease corresponding to the target of measurement perpendicularly. Reading from the ruler the distance from the point where the tip of the chord-playing finger being measured bends perpendicularly to it, or so close to perpendicular as possible without exceeding the limit of the perpendicular finger onto the side of the palm.
Measuring by the profile measurer 20 by setting the palm's crease corresponding to the plane being measured on the edge on the side of the hinge of the measuring plate and by ensuring that the wrist come toward the measuring plate 24 describing the plane of the fretboard at an angle following the item being measured the wrist being straight or at its maximal natural flexion. The chord-playing fingers are set linearly on the back of the measuring plate 24 such that the finger extending farthest on the plane of the fretboard 13 must lower itself on the measuring plate 24, or as close to perpendicular as possible without exceeding the limit of the perpendicular finger onto the side of the palm. Reading from the opposite side of the measuring plate the height point of the chord-playing finger from the edge on the side of the hinge to the centre point of the chord-playing finger's contact surface.
Measuring from the image shot from the side profile of the hand on the side of the thumb, wherein the tip of the finger reaching farthest has been set perpendicular against some kind of a side table operating as the plane of the fretboard 13 and the thumb and the other fingers are in such a position as relaxed as possible where they do not come in the way of the crease of the plane operating as the target being measured distinguishable of the side of the chord-playing finger of palm in the image. Measuring the distance from the palm's grease corresponding to the plane to the contact point of the tip of the finger and the plane.
Measuring the highest height point (OT1S2) of the finger which extends to the shortest from the palm plane OT1 when the wrist is straight
Measuring the highest height point (OT1F2) of the finger which extends to the shortest from the palm plane OT1 when the wrist is in its maximal natural flexion
Measuring the highest height point (OT2S2) of the finger which extends to the shortest from the palm plane OT2 when the wrist is straight
Measuring the highest height point (OT2F2) of the finger which extends to the shortest from the palm plane OT2 when the wrist is in its maximal natural flexion
Measuring by using the ruler by setting the ruler as if a plane of the fretboard which rises from the palm's plane from the crease corresponding to the target of measurement perpendicularly. Reading from the ruler the distance from the point where the chord-playing finger being measured sets at its highest.
Measuring by the profile measurer 20 by setting the palm's crease corresponding to the plane being measured on the edge on the side of the hinge of the measuring plate and by ensuring that the wrist come toward the measuring plate describing the plane of the fretboard at an angle following the item being measured the wrist being straight or at its maximal natural flexion. The chord-playing fingers are set linearly on the back of the measuring plate such that the finger reaching the shorted plane of the fretboard must settle on the measuring plate so far from the edge it can naturally reach. Reading from the opposite side of the measuring plate the point of the chord-playing finger from the edge on the side of the hinge to the centre point of the chord-playing finger's contact surface. The accuracy of the point can be ensured by bringing the other fingers to the linear position with it and, if needed, it can be adjusted a bit.
Measuring from the image shot from the side profile of the hand on the side of the thumb, wherein the finger reaching shortest has been set at its highest natural point against some kind of a side table operating as the plane of the fretboard 13 and the thumb and the other fingers are in such a position as relaxed as possible where they do not come in the way of the crease of the plane operating as the target being measured distinguishable of the side of the chord-playing finger of palm in the image. Measuring the distance from the palm's grease corresponding to the plane to the contact point of the finger and the plane.
The ranges of variation of the fingers set linearly in the width direction of the fretboard 13 are calculated with the following formulas:
range of variation on OT1 plane with straight wrist: OT1S2-OT1S1
range of variation on OT1 plane in wrist's maximal flexion: OT1F2-OT1F1
range of variation on OT2 plane with straight wrist: OT2S2-OT2S1
range of variation on OT2 plane in wrist's maximal flexion: OT2F2-OT2F1

Formulas on the effect of the wrist's flexion on the height points and ranges of variation of the linear chord-playing fingers at different fretboard planes:

$$linear cord - playing fingers ’ lowest height point in specified wrist ’ s flexion on OT 1 plane = ( OT 1 F 1 - OT 1 S 1 ) / ( amount of wrist ’ s flexion at point of OT 1 F 1 ) \times ( amount of wrist ’ s flexion ) + OT 1 S 1$$

$$l inear cord - playing fingers ’ highest height point in specified wrist ’ s flexion on OT 1 plane = ( OT 1 F 2 - OT 1 S 2 ) / ( amount of wrist ’ s flexion at point of OT 1 F 2 ) \times ( amount of wrist ’ s flexion ) + OT 1 S 2$$

$$linear cord - playing fingers ’ lowest height point in specified wrist ’ s flexion on OT 2 plane = ( OT 2 F 1 - OT 2 S 1 ) / ( amount of wrist ’ s flexion at point of OT 2 F 1 ) \times ( amount of wrist ’ s flexion ) + OT 2 S 1$$

$$linear cord - playing fingers ’ highest height point in specified wrist ’ s flexion on OT 2 plane = ( OT 2 F 2 - OT 2 S 2 ) / ( amount of wrist ’ s flexion at point of OT 2 F 2 ) \times ( amount of wrist ’ s flexion ) + OT 2 S 2$$

$$linear chord - playing fingers ’ range of variation in specific wrist ’ s flexion on OT 1 plane - linear chord - playing finger ’ s highest height point in specified wrist ’ s flexion on OT 1 plane - linear chord - playing fingers ’ lowest height point in specified wrist ’ s flexion on OT 1 plane$$

$$linear chord - playing fingers ’ range of variation in specific wrist ’ s flexion on OT 2 plane - linear chord - playing finger ’ s highest height point in specified wrist ’ s flexion on OT 2 plane - linear chord - playing fingers ’ lowest height point in specified wrist ’ s flexion on OT 2 plane$$

When shaping the neck profile, the musician's posture and the positioning of the stringed instrument in relation to the musician must be considered. In relation to the high register of the neck, the musician's palm must come on the neck of the stringed instrument at a specific angle and in a specific position in order for the chord-playing fingers be able to reach sufficiently well to play the bass strings. On the back of the neck, the thumb needs a sufficient income angle so that it would not be in too tight a space and lock the hand. If the forearm is in the flexion of about 90 degrees, which is the required minimum for many musicians' hands, and the lower edge of the neck 11 of the stringed instrument 10 is on the plane of about the palm, all flexion toward the neck 11 must come from the thumb's abduction and, in order for the playing not to be awkward, the abduction must be within its calculated limits or, otherwise, the hand and the fingers enter into a strained state. If the fingers do not reach to the bass strings in accordance with the total amount of flexion against the neck 11 and the thumb's abduction does not solve the situation, the options are few. The palm's plane being on the lower edge of the neck, the wrist cannot do the required flexion. Lowering the palm lower from the neck 11 by the forearm's extension and pushing slightly forward by the upper arm's flexion in order to create more space for the wrist bending toward the neck 11 are also often insufficient because, when playing from the high register, the reaching of the bass strings in this kind of arrangement is in such a situation about the amount of deviation required from the wrist in which the wrist's flexion changes the angle of deviation during the movement in the wrong direction in relation to the neck 11 considering the high register, and this is a feature of the wrist's flexion in a specific degree of deviation.

In order to have a better reach to the bass strings in the high register, the neck can be raised in relation to the musician, whereby the total degree of tilt toward the neck increases. A second option is to move the neck 11 more to the side toward the chord-playing hand 14, whereby changes, inter alia, at the point of the wrist's deviation and the upper arm's internal rotation better enable the wrist's flexion and, thus, better reach to the bass strings. A third option is to tilt the neck of the stringed instrument to a sharper angle, whereby the hand approaches the lower edge of the neck 11 from a different angle thus enabling a sharper total flexion and, thus, better reach to the bass strings. As a fourth option, or as an addition to the previous options, this invention enables the shaping of the neck profile such that the degree of flexion required of the upper limb toward the lower edge of the neck decreases. The thumb gets more space on the back of the neck and does not lock in the same way when playing, which enables the more relaxed movement of the chord-playing fingers. The additional space required by the thumb is taken into account in the profile of the neck 11 by means of the measurements, but this can be done by the principle of minimum adjustment.

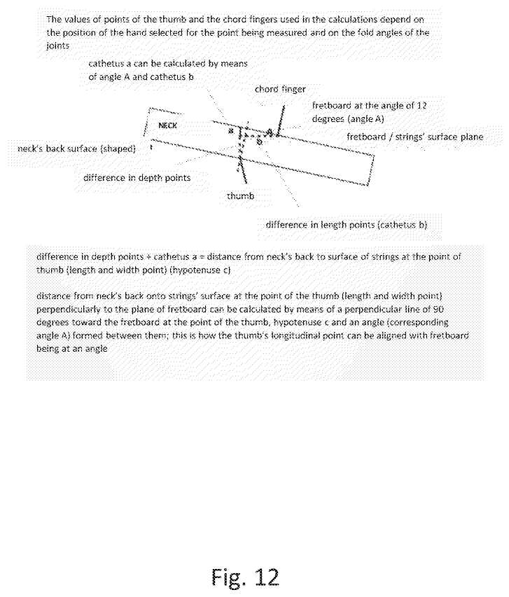

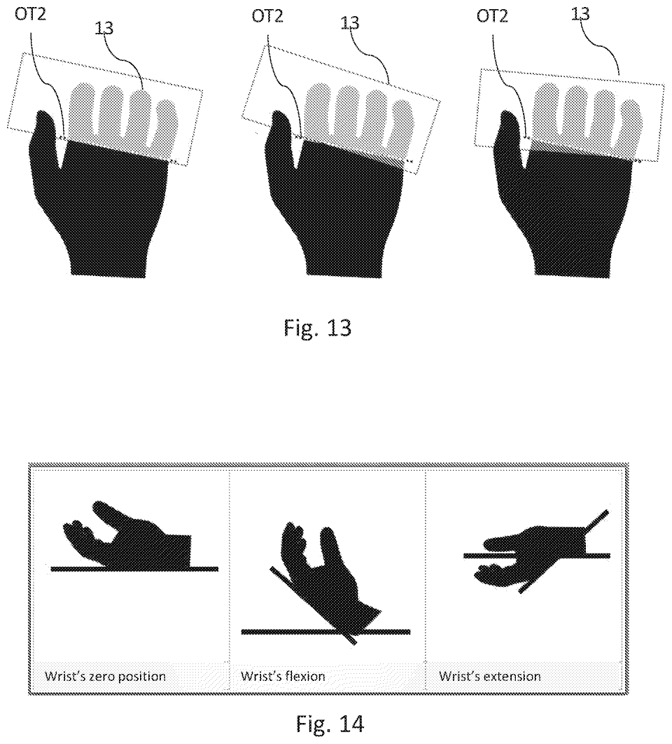

FIG. 12 is a schematic view of a cross-section of the neck and 11 the significance of the different dimensions in relation to the neck.

An advantage of the method and system according to the invention is that the neck profile, or a part of it, of the stringed instrument 10 can be measured without the stringed instrument directly from the musician's hand using different methods: by imaging, by scanning, by manual measurement, or in any way. By means of the profile measurer 20 designed to support the measuring method, accurate measurement results tied to each other always at the same stage.

Those skilled in the art will find it obvious that, as technology advances, the basic idea of the invention can be implemented in many different ways. The invention and its embodiments are thus not restricted to the above-described examples but may vary within the scope of the claims.

1. A method for shaping a neck of a stringed instrument, characterised by
measuring a musician's palm length in the direction of the forearm from the folding point of the wrist until the plane of the thumb's base;
measuring the musician's thumb length from the crease of the ulnar base to the tip of the thumb;
measuring the distance of the musician's thumb from the forefinger substantially in the direction of the musician's arm;
measuring the thumb's relaxed point after the combined flexion-abduction movement;
measuring the thumb's relaxed point after the combined extension-abduction movement;
measuring the distance in the direction of the forearm from the thumb's base to the plane of the creases of the chord-playing fingers' base joints;
measuring in the direction of the forearm the distance from the plane of the creases of the chord-playing fingers' base joints to the creases of the fingers' base members, where the fingers visibly separate from each other outside the rest of the palm;
calculating the maximum thickness of the neck of the stringed instrument at the point of the thumb being perpendicular to the fretboard by deducting the thumb's length from the distance between the thumb's base and the chord-playing fingers' bases on the palm at an angle in accordance with the thumb's relaxed point after the combined flexion-abduction movement.

2. A method according to claim 1, wherein:
calculating the maximum thickness of the neck of the stringed instrument at the point of the thumb being perpendicular to the plane of the fretboard by deducting the thumb's length from the distance between the thumb's base the chord-playing fingers' bases on the palm at an angle in accordance with the thumb's relaxed point after the combined flexion-abduction movement in the wrist's flexion.

3. A method according to claim 1, wherein:
calculating the maximum thickness of the neck of the stringed instrument at least in two points in the longitudinal direction of the neck, the forearm being at different angles in relation to the neck of the stringed instrument.

4. A profile measurer for measuring the maximum thickness of a neck of a stringed instrument, which comprises a hand support, a scale plate and a measuring plate, wherein the hand support is fastened to the scale plate substantially perpendicularly from the surface of the scale plate, and wherein the measuring plate is hinged either to the hand support or the scale plate such that the measuring plate is located rotatably in relation to the hand support.

5. A method according to claim 1, wherein the measurements are performed by said profile measurer.

6. A method according to claim 1, wherein a reference measure is set beside the hand such that the chord-playing hand and the reference measure are substantially at the same distance from the camera;
shooting images of said measures by the camera;
calculating normalised hand dimensions from the measurements obtained from the images in the scale of the reference measure;
calculating the dimensions of the profile of the neck of the stringed instrument.

7. A method according to claim 6, wherein shooting the images by a scanner by setting the chord-playing hand and the reference measure on the imaging table of the scanner.
